(19) United States
(12) Patent Application Publication
(10) Pub. No.: US 2021/0186819 A1
(43) Pub. Date: Jun. 24, 2021

(54) Dental Fracture Detection Compositions and Methods

(71) Applicant: Moore Dental Technologies and Solutions LLC, Chicago, IL (US)

(72) Inventor: Melissa S. Rebeles, Chicago, IL (US)

(73) Assignee: Moore Dental Technologies and Solutions LLC, Chicago, IL (US)

(21) Appl. No.: 17/274,091

(22) PCT Filed: Sep. 6, 2019
(86) PCT No.: PCT/US19/49929

§ 371 Date: Mar. 5, 2021

Related U.S. Application Data
(60) Provisional application No. 62/728,519, filed on Sep. 7, 2018.

(51) Int. Cl. A61K 6/25; A61K 49/04; A61K 51/02; A61K 6/17; A61K 47/44; A61K 47/10; A61K 9/00

(52) U.S. Cl. CPC A61K 6/25; A61K 49/0404; A61K 51/02; A61K 9/0063; A61K 47/44; A61K 47/10; A61K 9/0019; A61K 6/17

(57) ABSTRACT

In some aspects, this disclosure relates to compositions and/or methods for identifying or diagnosing dental fractures. In some examples, a composition including a contrast agent is applied to a tooth, and then a diagnostic image of that tooth is obtained. The composition may include barium sulfate and/or radioactive iodine, which may be present in or throughout a dispersion medium facilitating application of the composition to a tooth. The barium sulfate may have a reduced particle size to facilitate dispersion within the medium, and therefore application to a tooth/ligament. The composition may include one or more oils, flavors, dyes, or other additives such as surfactants. The composition may be applied topically or via injection, such as injection into the adjacent periodontal ligament.

### CROSS REFERENCE TO RELATED APPLICATIONS

This application claims the benefit of, and priority to, U.S. Patent Application No. 62/728,519, filed Sep. 7, 2018, the content of which is expressly incorporated herein by reference in its entirety for any and all non-limiting purposes.

### TECHNICAL FIELD

In some aspects, this disclosure relates to compositions and methods for use in identifying or diagnosing dental fractures, such as hairline fractures. In some examples, a composition including a contrast agent is applied to a tooth, and then a diagnostic image of that tooth is obtained. The composition may include barium sulfate and/or radioactive iodine, which may be present in or throughout a dispersion medium facilitating application of the composition to a tooth. The composition may be applied topically or via injection, such as injection into the adjacent periodontal ligament.

### BACKGROUND

Cracked and/or fractured teeth are one of the leading causes of tooth loss. While tooth fractures are known to cause serious dental problems, they are still difficult to diagnose using traditional methods. Macroscopic and symptom-driven diagnosis are the most common methods of diagnosis of fractured teeth but, often, visual examination is unable to identify a fracture at the early stages of the fracture.

Thus, fractures are often only diagnosed after the fracture has progressed to a significant degree, and there may be pulpal involvement and/or periodontal break-down. At this stage, significant treatment methods, such as dental implant tooth replacement, may be needed. Even in cases where more significant detrimental symptoms may not have occurred, the patient may have ongoing discomfort or pain from a smaller fracture that may escape diagnosis under traditional methods. Use of microscopic equipment can help identify fracture clues, but this requires the use of relatively high-level magnification devices that may not be readily available or easily afforded.

### SUMMARY

This Summary provides an introduction to some general concepts relating to this disclosure in a simplified form, where the general concepts are further described below in the Detailed Description. This Summary is not intended to identify key features or essential features of the disclosure.

In some aspects, the disclosure relates to compositions. The compositions may be used to detect and/or diagnose a dental fracture. The compositions may include one or more contrast agents and a dispersion medium. The one or more agents may be dispersed throughout the dispersion medium. In some examples, the dispersion medium includes propylene glycol. In certain examples, the dispersion medium is an aqueous solution, such as diluted propylene glycol. In some embodiments, the dispersion medium comprises one or more antibacterial materials. In various embodiments, the dispersion medium includes an alcohol.

In some embodiments, the one or more contrast agents include barium sulfate. The contrast agent(s), such as barium sulfate, may have a reduced particle size in whole, or in substantial amounts. In various examples, at least eighty percent of the barium sulfate has a particle size of about 10 microns or less, about 15 microns or less, about 8 microns or less, or about 6 microns or less. The barium sulfate may be milled to reduce its particle size. In certain embodiments, the barium sulfate is between about 0.1 to 30 percent by weight of the composition. In some embodiments, the barium sulfate is between about 0.2 to 20 percent by weight of the composition. In various examples, the barium sulfate is between about 6 to 7 percent by weight of the composition.

In certain examples, the composition included one or more flavors, oils, dyes, or a combination thereof. In some examples, these include lavender oil and/or clove oil. In various embodiments, the composition includes one or more surfactants.

In some examples, the one or more contrast agents include a radioactive contrast agent, such as a radioactive contrast agent including iodine. In certain embodiments, the dispersion medium has a viscosity between and about 5 and 80 cPs at 25 degrees Celsius.

In accordance with another aspect of the disclosure, processes are disclosed. In some examples, a process for detecting a dental fracture includes applying a composition to a tooth and taking a diagnostic image of the tooth, where the composition includes one or more contrast agents and a dispersion medium. In certain examples, the application of the composition is through an injection into the tooth's periodontal ligament. In some embodiments, the composition is applied to the tooth using a brush, swab, sponge, or tray. In various examples, the process further includes waiting a dispersion period, wherein the composition enters the tooth during the dispersion period. In certain examples of the process, the one or more contrast agents include barium sulfate, and in some examples, at least eighty percent of the barium sulfate has a particle size of about 10 microns or less.

These summary descriptions merely provide examples of materials, compositions, processes and/or process steps that may be performed in one or more embodiments. In certain embodiments, compositions and processes include additional combinations or substitutions. To that end, other details and features will be described in the sections that follow. Any of the features discussed in the embodiments of one aspect may be features of embodiments of any other aspect discussed herein. Moreover, additional and alternative suitable variations, features, aspects and steps will be recognized by those skilled in the art given the benefit of this disclosure.

These and other features, advantages, and objects of the present disclosure will be further understood and appreciated by those skilled in the art by reference to the following specification.

### DETAILED DESCRIPTION

In some aspects, the disclosure relates to compositions. The compositions may be used to detect and/or diagnose a dental fracture, for example through a diagnostic image such as an x-ray. The compositions may include one or more contrast agents and a dispersion medium. The medium may facilitate application of the contrast agent(s) to a tooth, such that a subsequent diagnostic image may indicate a fracture that is highlighted on the image through the contrast agent(s). For example, the contrast agent(s) may be a material that is visible and/or glows on a radiograph and/or other diagnostic image/result.

In some embodiments, the one or more contrast agents include barium sulfate, one or more an iodinated agent(s), or a combination thereof. In some examples, the one or more contrast agents include a radioactive contrast agent, such as a radioactive contrast agent including iodine. The one or more contrast agents are those safe for human use and/or consumption in the amounts needed for the dental fracture diagnosis process.

The contrast agent compositions may be applied to a tooth or gum area as a liquid, paste, solution, suspension, colloid, or emulsion. The contrast agents may have proprieties facilitating an application to tooth/gum areas. For example, in examples using barium sulfate as a contrast agent (whether in whole or in part), the barium sulfate may have a reduced particle size. Likewise, other contrast agents used instead of and/or in combination with barium sulfate may have a reduced particle size, including but not limited to the sizes discussed in reference to the barium sulfate example embodiments (for the avoidance of doubt, any other features, beyond particle size, discussed for the barium sulfate example composition/methods may similarly apply to other compositions/methods involving different and/or additional contrast agents). At relatively larger particle sizes, achieving a suitable fluid application is more difficult, as the suspension may separate and/or the barium sulfate may aggregate and/or become lumpy. Relatively larger barium sulfate particles sizes may result in a product that does not coat the tooth well. This results in an unpleasant experience for the patient. Larger particle sizes may also inhibit the barium sulfate's ability to penetrate a fracture, resulting in inconclusive or lower clarity radiograph results.

The particle size of the barium sulfate may be reduced to enhance performance in fracture diagnosis, for example through milling, shearing, crunching, grinding, and/or other mechanical processes (and including combinations of these processes, including sequences of two or more processes), such as processes using other components, like beads, discs, or spheres, for example Yttrium Stabilized Zirconia grinding beads, and/or metal or metallic alloy beads, or procedures involving rotors or other mixers, and homogenizers. In some examples, the barium sulfate (and/or other contrast agent) may be added to a mixer with one or more liquids, such as water, to facilitate wet milling of the contrast agent(s), which may facilitate reaching even smaller particle sizes. In some examples, a wetted contrast agent, paste, and/or slurry is acted upon by milling media, such as metal or ceramic material. In some examples, contrast agent(s) are initially processed (billed, sheared, and/or grinded etc.) in dry form, then wetted to be processed in one or more additional steps to achieve further reductions in size.

In various examples, around eighty percent or more of the barium sulfate has a particle size of about 15 microns or less, about 10 microns or less, about 8 microns or less, about 6 microns or less, about 5 microns or less, or about 3 microns or less. In certain examples, around ninety percent or more of the barium sulfate has a particle size of about 15 microns or less, about 10 microns or less, about 8 microns or less, about 6 microns or less, about 5 microns or less, or about 3 microns or less. In certain examples, around ninety-five percent or more of the barium sulfate has a particle size of about 15 microns or less, about 10 microns or less, about 8 microns or less, about 6 microns or less, about 5 microns or less, about 3 microns or less, or about 2.5 microns or less. In various examples, around ninety-nine percent or more of the barium sulfate has a particle size of about 15 microns or less, about 10 microns or less, about 8 microns or less, about 6 microns or less, about 5 microns or less, about 3 microns or less, or about 2.5 microns or less.

In various embodiments, around fifty percent or more of the barium sulfate has a particle size of about 8 microns or less, about 6 microns or less, about 5 microns or less, about 4 microns or less, about 3 microns or less, about 2 microns or less, about 1 micron or less, about 0.75 microns or less, or about 0.5 microns or less. In various embodiments, around thirty percent or more of the barium sulfate has a particle size of about 8 microns or less, about 6 microns or less, about 5 microns or less, about 4 microns or less, about 3 microns or less, about 1 micron or less, or about 0.5 microns or less.

In certain examples, around twenty percent or more of the barium sulfate has a particle size of about 5 microns or less, about 2 microns or less, about 1 micron or less, about 0.75 microns or less, about 0.5 microns or less, or about 0.25 microns or less. In some embodiments, around ten percent or more of the barium sulfate has a particle size of about 5 microns or less, about 2 microns or less, about 1 micron or less, about 0.75 microns or less, about 0.5 microns or less, about 0.4 microns or less, or about 0.25 microns or less. In various embodiments, around five percent or more of the barium sulfate has a particle size of about 5 microns or less, about 2 microns or less, about 1 micron or less, about 0.75 microns or less, about 0.5 microns or less, about 0.4 microns or less, or about 0.25 microns or less.

In certain examples, around five percent or more of the barium sulfate has a particle size of about 0.4 microns or less, about 0.35 microns or less, or about 0.25 microns or less.

In various examples, around 50 percent of more of the barium sulfate, or 60 percent or more, 70 percent or more, 80 percent or more, or 90 percent or more of the barium sulfate, has a particle size of ten microns or less. In various examples, around 50 percent of more of the barium sulfate, or 60 percent or more, 70 percent or more, 80 percent or more, or 90 percent or more of the barium sulfate, has a particle size of about five microns or less. In some examples, 1 percent or less of the barium sulfate has a particle size of 15 microns or more, or 5 percent or less has a particle size of 15 microns or more, or 10 percent or less. In certain examples, 1 percent or less of the barium sulfate has a particle size of 10 microns or more, or 5 percent or less has a particle size of 10 microns or more, or 10 percent or less.

In some embodiments, the barium sulfate has a mean particle size of about 5 microns or less, about 4.5 microns or less, about 4 microns or less, about 3.5 microns or less, about 3 microns or less, about 2 microns or less, about 1.5 microns or less, about 1.25 microns or less, or about 1 micron or less. In certain examples, the barium sulfate has a median size of about 4 microns or less, about 3 microns or less, about 2.5 microns or less, about 2 microns or less, about 1.5 microns or less, about 1 micron or less, or about 0.75 microns or less.

Reducing the particle size of one or more solid contrast agents, e.g. barium sulfate, for example through a wet milled, steel-ball process, may provide certain benefits to the composition. In some examples, relatively smaller contrast agent particle sizes may provide for better visibility in radiographs. In certain example, relatively smaller contrast agent particle sizes may provide a more pleasing consistency and/or texture of the composition for the patient, and/or on that is easier to apply from the perspective of the dental caregiver. In various embodiments, relatively smaller contrast agent particle sizes may enhance shelf life stability, for example by not settling and/or separating as quickly as other compositions with larger particle sizes more susceptible to gravitational effects over time, as the contrast agent may stay suspended for a longer period in the liquid composition mixture. In certain examples, relatively smaller contrast agent particle sizes disperse better within the composition and/or disperse better when applied to a patient. In various embodiments, relatively smaller contrast agent particle sizes reduce or eliminate the need for tumbling or agitation of the composition prior to application. Finally, in some examples, relatively smaller contrast agent particle sizes may provide a more pleasing visual appearance of the composition for the patient, for example by providing a visual appearance.

In certain embodiments, the barium sulfate is between about 0.1 to 30 percent by weight of the composition. In some embodiments, the barium sulfate is between about 0.2 to 20 percent by weight of the composition, between about 1 and 10 percent, between about 5 and 8 percent, or between about 5 and 10 percent, by weight. In various examples, the barium sulfate is between about 6 to 7 percent by weight of the composition. In some examples, the composition is about 6.25 percent by weight barium sulfate. In certain examples, the composition is about 20 percent or less barium sulfate, by weight, about 15 percent or less, about 10 percent or less, about 8 percent or less, about 6 percent or less, or about 5 percent or less. In various embodiments, the composition is about 2 percent or more barium sulfate, by weight, about 4 percent or more, about 6 percent or more, about 8 percent or more, about 10 percent or more, or about 12 percent or more.

In some examples, the composition includes a dispersion medium that contains one or more contrast agents. The dispersion medium may be a single material or may be a mixture of two or materials. The dispersion medium and/or composition may include one or more additives. In some examples the dispersion medium is a mixture of two or more primary materials, and one or more additives may be added to the composition and/or medium. For example, in some embodiments the dispersion medium is predominantly made up of two or more main materials (e.g. mixtures of propylene glycol, glycerin, and/or water) with smaller amounts of one or more additives optionally present. In some examples, the composition includes the one or more contrast agents and the dispersion medium.

The one or more contrast agents may be dispersed throughout the dispersion medium. In some examples, the dispersion medium includes propylene glycol. In certain examples, the dispersion medium is an aqueous solution, such as diluted propylene glycol. In some embodiments, the dispersion medium comprises one or more antibacterial materials, such as propylene glycol. In some embodiments, the dispersion medium comprises one or more antioxidant materials. In various embodiments, the dispersion medium includes an alcohol. In some examples, the dispersion medium may include or consist of one or more emulsifiers and/or surfactants, where these may assist in the dispersion of the one or more contrast agents. The dispersion medium may include one or more materials that have a perceptible flavor or sweetness to help make the one or more contrast agents more palatable for use in the patient's mouth, or may mask the flavor of the one or more contrast agents entirely (either through the flavor of the medium material(s) and/or due to one or more flavoring additives). The dispersion medium may include one or more materials that have other organoleptic properties, such as a thickening agent.

As representative examples, the dispersion medium may consist of or include one or more alcohols or polyols. In some examples, the dispersion medium may consist of or include one or more of propylene glycol, trimethylene glycol ether, glycerol, erythitol, sorbitol, mannitol. In some examples, the dispersion medium may consist of or include one or more edible polyols and/or edible alcohols such as sugar alcohols. The dispersion medium component(s) may be miscible with water. In some examples, the dispersion medium component(s) are mixed with and/or diluted with water, and optionally other medium components.

In some examples, the dispersion medium may include a component, such as a sugar alcohol or other sugar component, that results in a cooling sensation upon application to the patient's mouth, as dissolution of a component (e.g. the sugar) results in an endothermic reaction. This may make the application more pleasing to the patient, for example by diverting their attention from the presence of the contrast agent(s).

The dispersion medium may also include one or more additives, such as components added to influence one or more of the compositions flavor, smell, appearance, and/or functional properties. Example flavorants may be naturally derived or artificial in nature, and may include fruit extracts, such as those based on orange, strawberry, raspberry, kiwi, cherry, banana, apple or peach, or non-fruit materials such as mint, anise, and/or vanilla, or other flavors such as vegetable based flavors, chocolate, and so on. In some examples, the dispersion medium includes one or more oils, such as essential oils. The one or more additives may include or consist of natural ingredients. The additives (as well as the dispersion medium component(s)) should be substances that are safe for human consumption or use in the amounts utilized, for example an additive may be used in an amount that is safe, or a diluted version is utilized. In some examples, any additives are soluble or miscible in the primary component(s) of the dispersion media.

In some examples, the dispersion medium includes clove oil, lavender oil, peppermint oil and/or fruit oil(s) (such as but not limited to a citrus oil). In certain embodiments, one or more homeopathic oils are added to the dispersion medium (although any components noted as part of the dispersion medium may be added separately, such as an ingredient added after the one or more contrast agents and dispersion medium are mixed together).

In some examples, one or more essential oils and/or flavor oils are added. Examples include lavender oil, peppermint oil, tea tree oil, lemon oil, and/or orange oil. The composition may utilize menthol.

Other example additives include antioxidants, for example vitamin A, vitamin C, or vitamin E. In some examples, citric acid and/or sodium citrate is an additive. The composition may also include one or more perseverates.

In certain examples, the dispersion medium includes or more pigments, dyes, or colorants. This may provide a different ornamental appearance of the composition rom the white or “milky” appearance of other barium sulfate compositions.

In some embodiments, the dispersion medium and/or composition includes one or more sweeteners or flavorings, or other edible components to further alter the taste and/or other organoleptic properties of the composition, such as consistency or mouthfeel. For example, one or more flavor oils such as citrus oil or peppermint oil may be used. The dyes or colorants may be derived from one or more natural sources, such as vegetables, but may also be artificial colorants. Example colorants may include beta-carotene, beet extract, or grape skin extract.

In certain embodiments, the composition and/or dispersion medium includes one or more thickeners, declumping/anti-caking agents, such as one or more of silica/silicate compound(s), talc, stearate compound(s), flour(s), starch(es). Other examples include polysaccharides, starches, gums such as vegetable gums, gelatin, pectin, protein thickeners, alginates, and/or carrageenan.

In some examples, the composition and/or dispersion medium includes one or more volatile aromatic compounds. This may assist in providing a pleasant smell and/or “cooling” effect to the patent. Other example additions include one or more buffers, bulking agents, viscosity altering components, surfactants, or stabilizers. In some examples, the one or more additives (regardless of type) in total provide about 10% or less of the composition weight as a whole, or about 5% or less, or about 2.5% or less, or about 1% or less. In certain examples, any particular additive provides about 10% or less of the composition weight as a whole, or about 5% or less, or about 2.5% or less, or about 1% or less. This disclosure expressly contemplates that, in at least some embodiments, no additives are provided, and a usable composition is provided consisting only of the dispersion medium and the contrast agent. This disclosure also expressly contemplates that, in at least some embodiments, no additives are provided for the purposes of flavor, mouthfeel, or visual appearance.

In various embodiments, the dispersion medium has properties such that it flows well and can be readily applied to a patient's tooth or gum area. For example, use of propylene glycol as a dispersion medium ingredient helps counteract that the grittiness of a milled barium sulfate, facilitate application by injection, brushing, or other methods, such has those discussed below. In other examples, where an iodine contrast agent is used, the dispersion medium may have flow and viscosity properties suited for the selected contrast agent.

In certain examples, the dispersion medium includes or consists of one or more components having a viscosity of about 40 cP to 45 cP (all viscosities are for 300 degrees K unless otherwise noted). In some examples, the dispersion medium includes or consists of one or more components having a viscosity of about 1 to 100 cPs, about 1 to 10 cPs, about 5 to 10 cPs, about 5 to 15 cPs, about 5 to 50 cPs, about 5 to 80 cPs, about 10 to 50 cPs, or about 5 to 30 cPs. In some examples, the dispersion medium includes or consists of one or more components having a viscosity of about 5 cPs or more, about 10 cPs or more, about 15 cPs or more, about 20 cPs or more, about 30 cPs or more, about 40 cPs or more, about 50 cPs or more, or about 75 cPs or more. In some examples, the dispersion medium includes or consists of one or more components having a viscosity of about 75 cPs or less, about 50 cPs or less, about 40 cPs or less, about 25 cPs or less, about 15 cPs or less, about 10 cPs or less, or about 5 cPs or less. Any of the above viscosity values or ranges may also be suitable for the composition and/or dispersion medium as a whole. For example, the dispersion medium may have a viscosity of about 1 to 100 cPs, about 1 to 10 cPs, about 5 to 10 cPs, about 5 to 15 cPs, about 5 to 50 cPs, about 5 to 80 cPs, about 10 to 50 cPs, or about 5 to 30 cPs. In certain embodiments, the dispersion medium has a viscosity between and about 5 and 80 cPs at 25 degrees Celsius, between about 5 and 15 cPs at 25 degrees Celsius, between about 10 and 15 cPs at 25 degrees Celsius. In examples, the composition and/or medium are not so thick that the composition and/or medium does not run or only slowly runs/seeps at room temperature, so as to allow relatively quick applications to a patient. Waiting 15 minutes for a solution to seep past the periodontal ligament, for example, is somewhat undesirable, although still functional and a usable application of the compositions described herein.

In certain examples, the composition included one or more flavors, oils, dyes, or a combination thereof. In some examples, these include lavender oil and/or clove oil. In various embodiments, the composition includes one or more surfactants.

In accordance with another aspect of the disclosure, processes are disclosed. In some examples, a process for detecting a dental fracture includes applying a composition to a tooth and then taking a diagnostic image of the tooth, where the composition includes one or more contrast agents and a dispersion medium. In some examples, the process is performed when other diagnostic or procedures are unable to diagnose or fix a problem a patent is having, such as persistent pain from a tooth. The processes may be particularly advantageous to perform when a patient is going to receive an x-ray or other diagnostic image as part of their care, as this will allow additional information to be determined via the diagnostic image. Thus, the process may be used in conjunction with other procedures like a root canal/crown application (for example, during the procedure before the crown/bridge is fixed, and/or at the time of replacement), or in diagnostic treatment methods for conditions such as a force trauma injury to the mouth/face, tooth pain, the presence of other sensations from a tooth, or other symptomatic conditions (such as patient symptomatic of hairline fracture, but where such a fracture cannot easily be diagnosed via traditional methods). As shown by the example noted later in the application, the process may also be useful and/or helpful when a patient's problems have persistently evaded diagnosis or treatment, including when a tooth removal and a dental implant are recommended.

In certain examples, the application of the composition is through an injection. The injection may be to the gum area surrounding the tooth where a diagnosis is desired, for example an injection into the tooth's periodontal ligament. When the composition is injection, the procedure may be followed by or concluded with a flushing procedure of another substance, such as saline or tap water. When injected the composition, and therefore the one or more contrast agents, may penetrate to tooth such that it will be easily visible in a diagnostic image. In example methods applying the composition via injection, a thinner, less viscous composition may facilitate the application. For example, compositions based on radioactive iodine, or a barium sulfate composition with a relatively less viscous dispersion medium (e.g. diluted propylene glycol, or a relatively lower viscosity alcohol) and/or a relatively lower amount of barium sulfate and/or relatively smaller barium sulfate may be suitable.

In some embodiments, the composition is applied topically to the tooth area, for example directly to the tooth, e.g. using a brush, swab, sponge, or tray. Thicker compositions or those with relatively higher amounts of contrast agents may be suitable for certain topical applications. For example, a thicker composition with 20% by weight barium sulfate may be applied to a crown to allow diagnosis of possible issues with the crown, but may be too thick to seep into the tooth to help diagnose other fractures. Smaller amounts and/or thinner solutions may facilitate travel of the composition, and therefore diagnosis of additional fractures and/or smaller fractures. But the specific conditions of the patient and the applicable dental procedures may also influence the topical application. For example, during a root canal, even thicker compositions may be easily applied. For certain compositions, additional amount of water or other wetting agent may need to be used to help apply the composition topically in the desired location.

As an example application, a dental patient may have the tooth area to be diagnosed dried, and then one or more barriers may be provided to prevent saliva contamination (such as gauze and/or cotton molds). Components such as cheek guards and/or cotton roll barriers under the tongue and/or on the buccal/facial side of the patient's mouth may also help prevent unnecessary ingestion. The area may also be continuously suctioned, for example with low volume suction, to further mitigate potential saliva contamination and/or unnecessary ingestion.

The composition may be applied topically, for example by using a plunger driving syringe with an application tip, such as a curved application tip having an aperture where the liquid composition is deposited or squirted onto the desired area, and/or a brush applicator tip where the brush materials/bristles receive the liquid composition, which is then applied by brushing onto the tooth. Other applications tips may also be used. The composition may also be applied through use of intraoral trays. The composition may be premixed or may be mixed at or near the site of application. The composition may be applied on the tooth and/or around the tooth (e.g. part or all of the adjacent gum area, including but not limited to the periodontal ligament), and/or may be brushed into the tooth, and/or around the tooth. The tooth may be partially coated or fully coated, and the coating may include the interproximal areas. The composition may be applied to infiltrate the surrounding gingiva, or may be injected inter marginally, to help insure sub gingival penetration. A dispersion period, such as a 60-90 seconds waiting period, where the patent may be upright, may then be allowed to made. Then, a dental professional may take the diagnostic image, e.g. an x-ray (e.g. a PA and/or BWX x-ray) or a CAT scan.

In some examples, the composition may be applied as part of a preventive treatment, for example to diagnose fractures early in the process so that more severe effects may be prevented entirely, or as part of a new patient protocol, or a procedure to be included whenever a patient receives one or more x-rays in the course of their typical care. In some examples, a tray shaped and sized to be placed around one or more teeth (such as all of the patient's upper or lower teeth) may be placed in a patient's mouth with the composition within the forming tray (while in others the tray(s) may be placed around all teeth). In this manner, the tray application may provide contact for all teeth in an area (or just one tooth), such that an x-ray or series of x-rays (e.g. a panoramic x-ray set) may all be focused on teeth that would indicate any fractures due to the applied one or more contrast agents. Depending on the severity of any detected fractures, a dental professional may recommend treatment, or may simply observe the fracture as needed in future appointments. This may assist patient's plan for and anticipate dental issues and/or any related costs for treatment. For example, the composition may assist in determining and/or locating hairline fractures, the orientation of the fracture, potential depth of the fracture, approximate length of the fracture.

In this manner, the compositions and/or process described herein may provide several benefits over traditional methods by facilitating relatively quick and/or inexpensive diagnostic methods. Accurate hairline fracture diagnosis could provide a true diagnosis and therefore allow a dentist and patient to make informed treatment choices. For example, greater accuracy in diagnosis may indicate that a fracture is not yet be at a point where an immediate extraction, grafts, or implants are necessary, but instead may be treated with other procedures, such as a restoring the tooth and treating with a crown, or not at all for the time being. This may allow a patient to plan and/or save appropriately for procedures that may be required at a later date, particularly if the accuracy of diagnosis allows a timeline of likely tooth life. Accurate diagnosis may also facilitate patient insurance claims and/or reimbursements. Even the most sophisticated method for diagnosing dental fractures rely on high-powered microscopic devices, which may not be readily available or easily afforded by all dental professions.

In various examples, the process further includes waiting a dispersion period. The composition may enter or penetrate the tooth during the dispersion period. The dispersion period should be sufficient to allow the contrast agent to travel, seep, penetrate, and/or traverse the tooth area such that any fractures will be visible on the subsequent diagnostic image. In some examples, the dispersion period is about 15 seconds or more, about 30 seconds or more, about 60 seconds or more, or about 90 seconds or more. In certain examples, the dispersion period is about 15 to 90 seconds, or about 60 to 90 seconds, or about 30 to 60 seconds. The dispersion time may coincide with the amount of time the composition, based on its viscosity and/or other properties, may need to penetrate or seep past the periodontal ligament, or travel within the tooth and/or travel within a fracture from the exterior of the tooth. In this manner, the complete process for the diagnosis of a dental fracture may be completely quickly. In some examples, the entire process, through the acquisition of a radiograph (or other image/result) may take three minutes or less, or two minutes or less.

After application, the area may be rinsed or flushed, for example with water. The composition may be applied in any suitable matter, but use of a topical syringe, injection, brush, trays, or direct application via a gloved finger may be the easiest for the dental professions. The compotation may be applied via a swab, a plunger, a brush, a sponge, a spray, an adhesive strip, a syringe or other injection device (but also including syringes such as plunger syringes not intended for injection into the gums or other body parts, but rather targeted deposition), a tray or other component (e.g. a film or strip of material) allowing one or more teeth to soak in or otherwise be placed into contact with the compositions. The composition may also be applied via an intraoral solution, such as a rinsing solution that leaves a film on oral features. The compositions may be stored as a mixed composition, for example in batches, or in smaller containers, such as a small sealed containers having an amount for an individual dose (enough for one tooth, enough for one patient's set of teeth, etc.), or may be stored as separated ingredients that are mixed on site for real time applications.

Processes of forming the composition may include treating the one or more contrast agents, forming the dispersion medium, and mixing these components. For example, barium sulfate may be milled and/or declumped using a milling process such as wet milling, or a mortar and pestle. The dispersion medium may be selected (e.g. propylene glycol may be used, or a dilute version may be selected), and any additives, such as any oil(s) and dye(s), may be added to make a pre-mixed dispersion medium and additive mix. The barium sulfate may then be mixed in a small amount into the premix, and may be added iteratively. Additional dispersion medium and/or dispersion medium and additive pre-mix may be added to adjust the composition's concentration of contrast agents.

### Example Formulations and Applications

As one example of a diagnosis method, and an illustration of the benefits of an example composition and method, a patient had dental issues for approximately two and half years that had been undiagnosed, despite efforts of multiple specialists and use of traditional radiograph techniques. An example composition, including 6.25% by weight barium sulfate, and the remaining weight was propylene glycol with small amounts of clove oil, lavender oil, and commercial food dye (these materials were pre-mix and the barium sulfate was added), was applied topically to the tooth at issue, and the subsequent radiograph showed a vertical fracture. This allowed the creation of a treatment plan, whereas before this diagnosis, the patient was going to have the tooth removed. As illustrated in this example, compositions and methods of this disclosure may allowed diagnosis, for example through radiographic imaging, of fractures that evade diagnosis from traditional dentistry techniques, such as traditional radiographs, including radiographs without any contrast agent(s).

Table 1 below provides example data for barium sulfate used in examples of the disclosure. In this example, the dry and wet milled barium sulfate was prepared using 0.8 mm YTZ as the grinding material. Approximately 20 grams of YTZ and approximately 10 grams of unground barium sulfate were added to the mixer, and in the wet milling examples approximately 15 grams of liquid pre-mix (in this example, primarily based on propylene glycol but also including small amounts of clove oil, lavender oil, and dye) as also added. Six cycles of approximately thirty seconds, at about 2000 rpm, were run (but, in other examples of the disclosure, different numbers of cycles (although multiple cycles can help provide reduced particle size, such as two or more cycles, or four or more cycles), and/or different cycle intervals, and/or different rpms may be used). In the dry milling process, the YTZ was then screened out of the resulting mixture. For the wet milling examples, an additional seven cycles of approximately one hundred and eighty seconds at about 3500 rpm were run, and then the YTZ was screened out. Measurement data was determined via a scanning electron microscope.

TABLE 1

| | Mean | D₅ | D₁₀ | D₅₀ | D₉₀ | D₉₉ |
|---|---|---|---|---|---|---|
| Material | (microns) | (microns) | (microns) | (microns) | (microns) | (microns) |
| BaSO₄ Commercial | 5.27 | 1.00 | 1.54 | 4.62 | 9.95 | 16.15 |
| Grade (hereinafter | | | | | | |
| Comparative Example) | | | | | | |
| Ground BaSO₄ (mortar | 4.51 | 0.44 | 0.81 | 3.74 | 9.20 | 15.20 |
| and pestle) (hereinafter | | | | | | |
| Example 1) | | | | | | |
| Ground BaSO₄ (mortar | 4.59 | 0.36 | 0.69 | 3.97 | 9.20 | 14.73 |
| and pestle) in Pre-Mix | | | | | | |
| (hereinafter Example 2) | | | | | | |
| Dry Milled Ground | 3.46 | 0.46 | 0.71 | 2.40 | 7.66 | 14.61 |
| BaSO₄ (hereinafter | | | | | | |
| Example 3) | | | | | | |
| Wet Milled Ground | 1.58 | 0.32 | 0.39 | 0.87 | 2.54 | 16.53 |
| BaSO₄ (hereinafter | | | | | | |
| Example 4) | | | | | | |

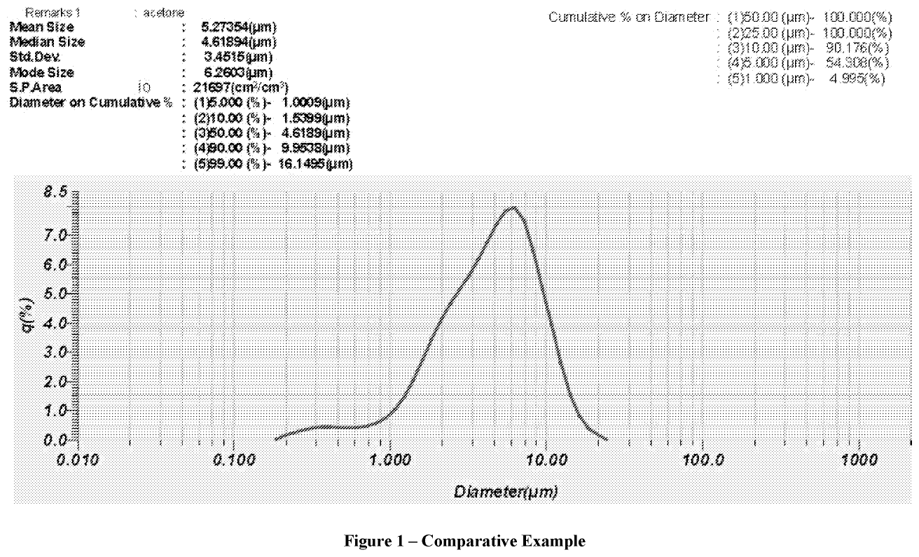

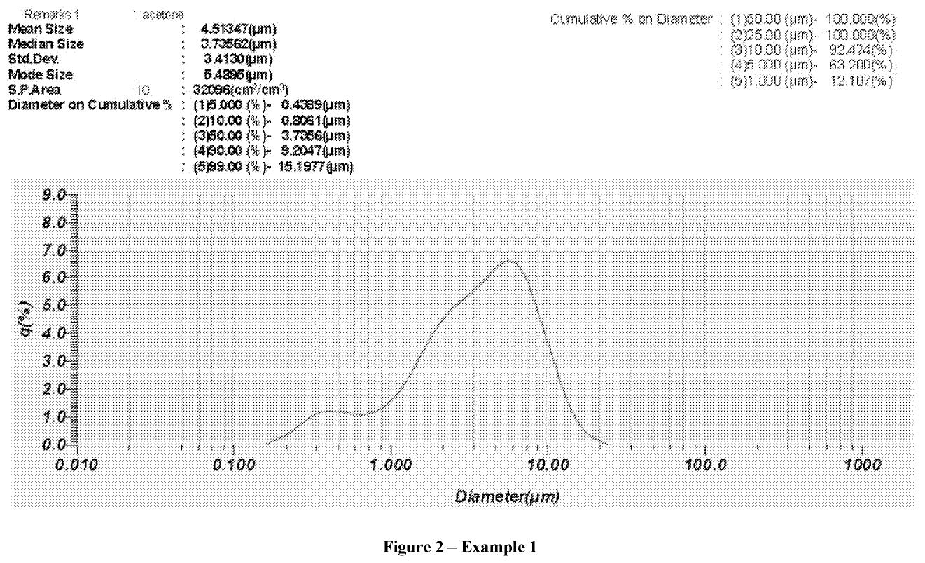

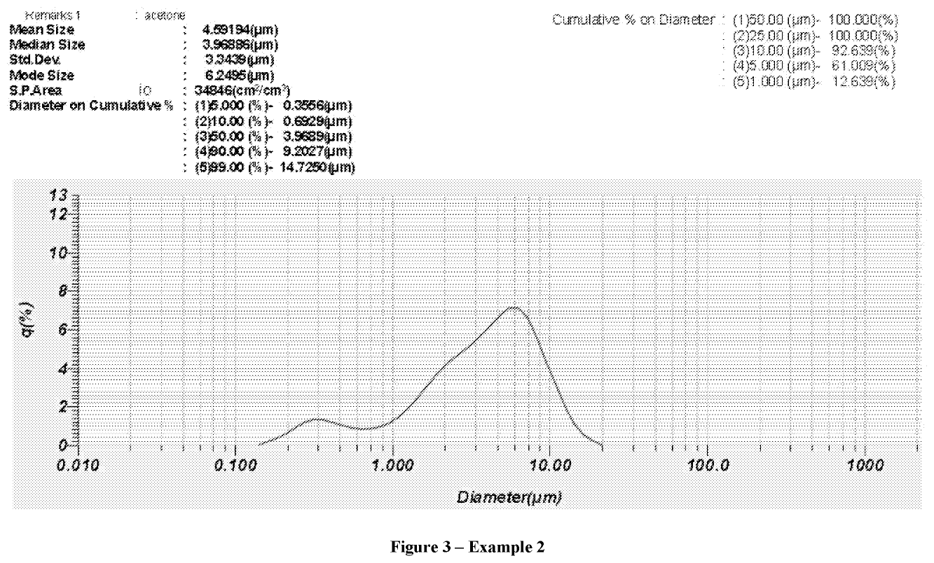

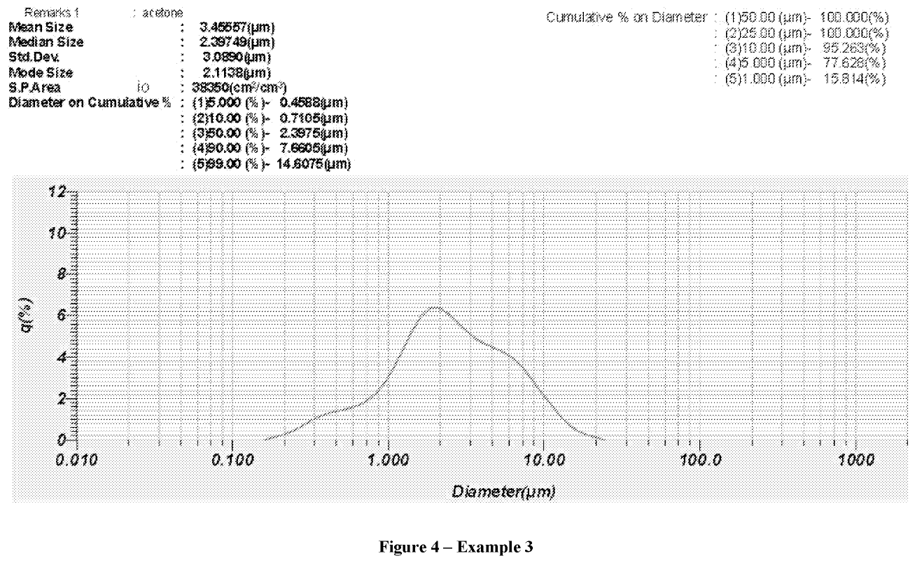

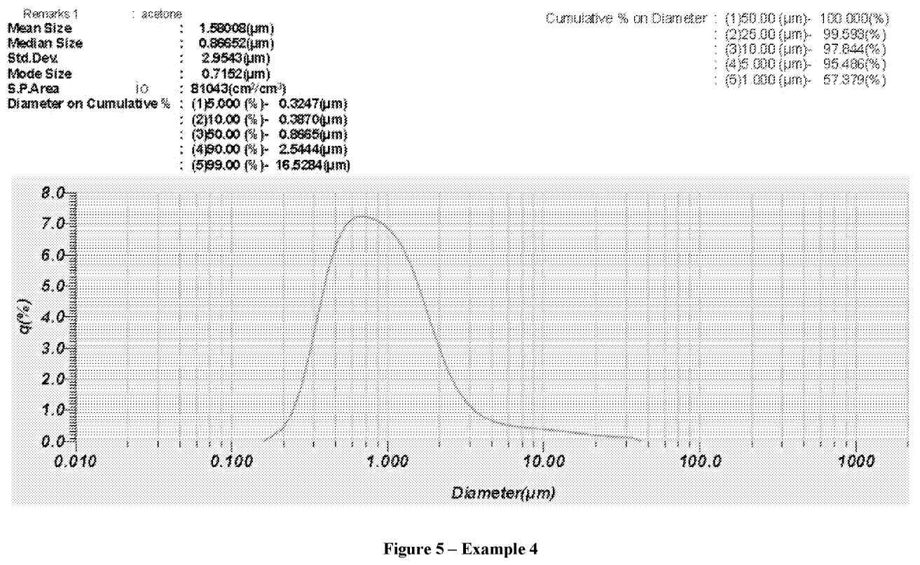

FIGS. 1-5 provide more detailed particle size information, where each figure is labeled to correspond with an example noted above. As illustrated here, one may obtain low particle sizes with milling processes, including sonication, ultrasonication, micro-grinding, and wet-milling, although, with sufficient effort, a mortar and pestle may also achieve low particle sizes. As also illustrated here, in some examples of the disclosure, a majority of the barium sulfate is less than one micron in particle size. As also illustrated here, in some examples of the disclosure, ten percent or less of the barium sulfate has a particle size of more than 2.5 microns.

These materials, compositions and system descriptions are merely examples. In certain embodiments, the materials, compositions, systems include additional combinations and/or substitutions of some or all of the features, materials, and/or components described above. Moreover, additional and alternative suitable variations, forms and components for the materials, compositions, and systems will be recognized by those skilled in the art given the benefit of this disclosure. Any of the features or materials described herein regarding materials, compositions, or systems may be utilized or incorporated into other materials, compositions or systems.

This disclosure also merely provides examples of the processes and/or process steps that may be performed in one or more embodiments, and additional and alternative suitable variations, steps, and combinations of steps will be recognized by those skilled in the art given the benefit of this disclosure. Finally, any of the features discussed in the example embodiments of the processes may be features of embodiments of the materials, compositions or systems (or components thereof), and vice versa.

1. A composition comprising:
one or more contrast agents; and
a dispersion medium.

2. The composition of claim 1, wherein the dispersion medium comprises propylene glycol.

3. The composition of claim 1, wherein the one or more contrast agents comprise barium sulfate.

4. The composition of claim 3, wherein at least eighty percent of the barium sulfate has a particle size of 10 microns or less.

5. The composition of claim 3, wherein the barium sulfate is between 0.2 to 20 percent by weight of the composition.

6. The composition of claim 5, wherein the barium sulfate is between 6 to 7 percent by weight of the composition.

7. The composition of claim 1, wherein the composition further comprises one or more flavors, oils, dyes, or a combination thereof.

8. The composition of claim 7, wherein the one or more flavors, oils, dyes, or a combination thereof comprise lavender oil and clove oil.

9. The composition of claim 3, wherein the barium sulfate has been milled to reduce particle size.

10. The composition of claim 1, wherein the dispersion medium comprises an antibacterial material.

11. The composition of claim 1, wherein the dispersion medium comprises an alcohol.

12. The composition of claim 1, further comprising one or more surfactants.

13. The composition of claim 1, wherein the one or more contrast agents comprise a radioactive contrast agent.

14. The composition of claim 13, wherein the radioactive contrast agent comprises iodine.

15. The composition of claim 1, wherein the dispersion medium comprises a viscosity between and 5 and 80 cPs at 25 degrees Celsius.

16. A process for detecting a dental fracture comprising:
applying a composition to a tooth; and
taking a diagnostic image of the tooth,
wherein the composition comprises one or more contrast agents and a dispersion medium.

17. The process of claim 16, wherein the application of the composition is through an injection into the tooth's periodontal ligament.

18. The process of claim 16, wherein the composition is applied to the tooth using a brush, swab, sponge, or tray.

19. The process of claim 16, further comprising waiting a dispersion period, wherein the composition enters the tooth during the dispersion period.

20. The process of claim 16, wherein the one or more contrast agents comprise barium sulfate.

21. The process of claim 20, wherein at least eighty percent of the barium sulfate has a particle size of 10 microns or less.
